(19) United States
(12) Patent Application Publication
(10) Pub. No.: US 2026/0263130 A1
(43) Pub. Date: Sep. 10, 2026

(54) SURGICAL CLAMP

(71) Applicant: VIORTEC PTY LTD., Weetangera (AU)

(72) Inventors: Thomas WARD, Griffith (AU); James MACDOUGALL, Prahran (AU)

(21) Appl. No.: 19/165,617

(22) PCT Filed: Mar. 15, 2024
(86) PCT No.: PCT/AU2024/050234

§ 371 Date: Sep. 16, 2025

(30) Foreign Application Priority Data
Mar. 16, 2023 (AU) 2023900720

(51) Int. Cl. A61B 17/88; A61B 17/00; A61B 90/00

(52) U.S. Cl. CPC A61B 17/8866; A61B 90/06; A61B 2017/0046; A61B 2090/064

(57) ABSTRACT

A surgical clamp for providing a stable mechanical grasp and stabilisation of bone during an orthopaedic surgical procedure, comprising: a first arm comprising: an extended section comprising a mount at the distal end thereof; and a first jaw portion located at the distal end of the first arm; a second arm comprising: a fastening platform located at the proximal end of the second arm; and a second jaw portion located at the distal end of the second arm; engagement means comprising a bolt to engage the first arm with the second arm about a pivot point formed by the engagement means to provide relative rotation of the first and second arms between: an open configuration whereby first jaw portion and second jaw portion are urged apart; and a clamped configuration whereby first jaw portion and second jaw portion are urged into clamping engagement with the patient's bone in use during the surgical procedure; a fastening component comprising a fastening bolt and a fastening nut, adapted to engage the first and second arm into a locked clamped configuration about the bone of a patient during the surgical procedure; and a handle adapted to be gripped by a surgical assistance for stabilisation of the patient's joint or bone during the surgical procedure.

### FIELD OF THE INVENTION

The present invention relates to a surgical device and in particular to a surgical clamp to assist a surgeon during an orthopaedic surgical procedure and which optionally provides real-time measurement of handling forces applied by the surgeon during the orthopaedic surgical procedure.

The invention has been developed primarily as a surgical clamp for use in ligament balancing procedures during knee arthroplasty surgery and will be described hereinafter with reference to this application. However, it will be appreciated that the invention is not limited to this particular field of use, but rather is further adaptable to alternate orthopaedic surgical procedures.

### BACKGROUND

Any discussion of the background art throughout the specification should in no way be considered as an admission that such background art is prior art, nor that such background art is widely known or forms part of the common general knowledge in the field in Australia or worldwide as at the priority date of the present application.

All references, including any patents or patent applications, cited in this specification are hereby incorporated by reference, which means that it should be read and considered by the reader as part of this text. That the document, reference, patent application or patent cited in this text is not repeated in this text is merely for reasons of conciseness.

Total knee replacement surgery is performed to replace worn or damaged knee joints that cause relentless pain and mobility issues to patients. This can be caused by a range of conditions of which severe osteoarthritis is the most common. Throughout recent years, more surgeons are beginning to use modern and advanced tools to help improve the ease and accuracy of the knee replacement process. This has removed a large amount of the inaccuracy and guesswork from the surgery, but one key step, the ligament balancing process, remains heavily reliant on the experience and ‘feel’ of the surgeon. This concerns creating a rectangular and equal gap in flexion and extension that can be mediated by adjusting tension in remaining ligaments, primarily between the medial collateral ligament (MCL) and lateral collateral ligament (LCL), or by adjusting the amount of bone resected from the tibia or femur.

Ligament balancing is a key step in the surgical operation where the implanted prosthesis is positioned to replicate the same ligament flexibility and tension as existed, for example, in the patient's knee or elbow joint prior to replacement surgery. Each patient will display slightly different measures in ligament flexion, due to the differing morphology of the human body between patients. Thus, it is impossible to apply a ‘one size fits all’ approach to all of the surgeon's arthroplasty replacement patients. Ligament balancing is a very important step in arthroplasty surgery to restore function to a patient's joint. For example, in knee arthroplasty, following surgical insertion of the joint implants, the surgeon manipulates the leg to qualitatively evaluate the tightness of the ligaments which surround the artificial joint, and performs further surgical adjustments if required. Ligament balancing aims to ensure that the tightness of the artificial joint resembles that of the patient's natural knee. Incorrect ligament balancing can result in joint dysfunction, discomfort, reduced mobility, reduced stability and poorer clinical outcomes for patients. It is estimated that nearly half of all knee revisions can be attributed to a cause that may be prevented with correct ligament balancing during the initial surgery. Regardless of the need for eventual revision, poor ligament balancing will result in a poor surgical outcome for the patient characterised by limited function of the replaced knee and ongoing pain for the patient.

Current surgical practice for ligament balancing involves multiple people during the surgery, guesswork, and uncomfortable positioning for the surgeon. In current surgical practice, for example during a total knee replacement (TK) surgery, an assistant will restrain the patient's upper leg, typically by manually embracing the patient's thigh, whilst the surgeon will estimate the full flexion of the ligaments by applying twisting and rotating actions to the lower leg, in order to qualitatively assess the tightness of the knee ligaments. The surgeon will qualitatively appraise the state of the ligaments by tactile feedback and this will inform the angles of the cuts the surgeon will make to the bone to accommodate the implants to the knee joint. The accuracy of this step is a significant factor in the surgical procedure as incorrect positioning can cause post-operative pain, restrict visual observations of the mobility and stability of the joint which can lead to longer recovery times for patients. However, the ligament balancing step of the procedure is highly reliant on the practitioner's training and experience and prone to systematic errors such are for example the resistive forces that the assistant is able to provide to retain the upper leg in a rigid position whilst the surgeon assesses the natural tension and positioning of the patient's ligaments, s

Current methods and tools available to orthopaedic surgeons do not enable a rigid grasp of the leg during ligament balancing and thus limits the accuracy with which the ligament tension can be qualitatively assessed. The assistant's hold/grip of the patient's soft tissue during the ligament balancing procedure is subject to variations in hold rigidity and introduces possible errors in the practitioner's appraisal of the state of the ligaments. There are currently no methods or tools which can quantitatively measure the force applied by the surgeon to the leg as they perform this procedure. Accumulated quantitative data on forces exerted during the test would not only improve patient outcomes, but increase repeatability to simplify training for this critical surgical step.

Those skilled in the art will appreciate that the invention described herein is susceptible to variations and modifications other than those specifically described. The invention includes all such variation and modifications. The invention also includes all of the steps, features, formulations, and compounds referred to or indicated in the specification, individually or collectively and any and all combinations of any two or more of the steps or features.

### SUMMARY

It is an object of the present invention to overcome or ameliorate at least one or more of the disadvantages of the prior art, or to provide a useful alternative.

Disclosed herein is a surgical clamp and method of use of a surgical clamp for providing quantitative and qualitative ligament balancing data during knee arthroplasty surgery, and to provide surgical stabilisation through firm holding of bone and the provision of quantitative ligament balancing data during knee arthroplasty surgery wherein the clamp comprises: a first and second arms comprising first and second jaw portions located at the distal end of respective arms; an engagement means comprising a bolt to engage the first with the second arm about a pivot point formed by the engagement means to provide relative rotation between: an open configuration whereby first jaw portion and second jaw portion are urged apart and a clamped configuration; and a clamped configuration whereby first jaw portion and second jaw portion are urged into clamping engagement with the patient's femur in use during a knee arthroplasty surgery; a locking bolt and a locking nut, adapted to engage the first and second arm into a locked clamped configuration about a femur of a patient during the knee arthroplasty surgery.

According to a first aspect of the invention, there is provided a surgical clamp for providing a stable mechanical grasp and stabilisation of bone during an orthopaedic surgical procedure.

The surgical clamp may comprise a first arm. The first arm may comprise an extended section comprising a mount at the distal end thereof. The first arm may further comprise a first jaw portion located at the distal end of the first arm.

The surgical clamp may further comprise a second arm. The first arm may comprise a fastening platform located at the proximal end of the second arm. The first arm may further comprise a second jaw portion located at the distal end of the second arm.

The surgical clamp may further comprise engagement means. The engagement means may comprise a bolt to engage the first arm with the second arm about a pivot point formed by the engagement means to provide relative rotation of the first and second arms. The engagement may be adapted for relative motion of the first and second arms to an open configuration whereby first jaw portion and second jaw portion are urged apart. The engagement may be further adapted for relative motion of the first and second arms to a clamped configuration whereby first jaw portion and second jaw portion are urged into clamping engagement with the patient's bone in use during the surgical procedure.

The surgical clamp may further comprise a fastening component. The fastening component may comprise a fastening bolt and a fastening nut. The fastening component may be adapted to engage the first and second arm into a locked clamped configuration about the bone of a patient during the surgical procedure.

The surgical clamp may further comprise a handle. The handle may be adapted to be gripped by a surgical assistance for stabilisation of the patient's joint or bone during the surgical procedure.

According to a particular arrangement of the first aspect, there is provided a surgical clamp for providing a stable mechanical grasp and stabilisation of bone during an orthopaedic surgical procedure, comprising:
a first arm comprising:
   an extended section comprising a mount at the distal end thereof; and
   a first jaw portion located at the distal end of the first arm;
a second arm comprising:
   a fastening platform located at the proximal end of the second arm; and
   a second jaw portion located at the distal end of the second arm;
engagement means comprising a bolt to engage the first arm with the second arm about a pivot point formed by the engagement means to provide relative rotation of the first and second arms between:
   an open configuration whereby first jaw portion and second jaw portion are urged apart; and
   a clamped configuration whereby first jaw portion and second jaw portion are urged into clamping engagement with the patient's bone in use during the surgical procedure;
a fastening component comprising a fastening bolt and a fastening nut, adapted to engage the first and second arm into a locked clamped configuration about the bone of a patient during the surgical procedure; and
a handle adapted to be gripped by a surgical assistance for stabilisation of the patient's joint or bone during the surgical procedure.

The handle may comprise a sensor unit adapted to releasably engage the mount and configured to sense reactive forces applied to the bone through the surgical clamp adapted for measuring quantitative force exchange data during an orthopaedic surgical procedure and to output the force exchange data.

The sensor unit may comprise a multiaxial sensor adapted to provide quantitative reactive force and/or torque measurements, such as may be generated during an intermediate procedure of the surgical procedure. The intermediate procedure may be a ligament rebalancing procedure. The sensor unit may further comprise a fixture component to ensure the sterile state of non-mechanical components including the said multiaxial sensor.

The fixture component may comprise a pair of ribbed plates. The fixture component may further comprise a securing component adapted to releasably engage the pair of ribbed plates. The securing component may comprise a sterile collar.

The handle may be interchangeable.

An inner face of the first and second jaw portions may be adapted to approximately match the shape of the patient's bone. An inner face of each of the first and second jaw portions may be conditioned with a surface to facilitate secure clamping engagement with the patient's bone. The conditioning may comprise contouring of the said inner face.

The first arm may comprise a receptacle located at the proximal end thereof. The second arm may comprise a fastening platform at the proximal end thereof comprising an elongate slot. The proximal end of the fastening bolt may comprise a stop adapted to engage the receptacle. The distal end of the fastening bolt may be adapted to pass though the elongate slot. The fastening nut may be adapted to engage the fastening bolt with the fastening platform thereby to secure the surgical clamp into a clamped locked configuration.

The sensor unit may provide output data quantitatively representative of the forces and torques applied to the stabilised bone during surgical procedure.

The surgical procedure may be knee arthroplasty surgery and the patient's bone may be the femur. The surgical procedure may be elbow arthroplasty surgery and the patient's bone may be the humerus.

According to a second aspect of the invention, there is provided an active surgical clamp for providing a stable mechanical grasp of bone and simultaneously measuring quantitative force exchange data during an orthopaedic surgical procedure. The active surgical clamp may comprise the surgical clamp of the first aspect. The active surgical clamp may further comprise a sensor unit adapted to releasably engage the mount and configured to sense reactive forces applied to the bone through the surgical clamp adapted for measuring quantitative force exchange data during an orthopaedic surgical procedure and to output the force exchange data.

According to a particular arrangement of the second aspect of the invention, there is provided an active surgical clamp for providing a stable mechanical grasp of bone and simultaneously measuring quantitative force exchange data during an orthopaedic surgical procedure, comprising: the surgical clamp of the first aspect; and a sensor unit adapted to releasably engage the mount and configured to sense reactive forces applied to the bone through the surgical clamp adapted for measuring quantitative force exchange data during an orthopaedic surgical procedure and to output the force exchange data.

According to a third aspect of the invention, there is provided a method for determining knee ligament characteristics for a ligament balancing data during a surgical procedure.

The method may comprise the step of providing a surgical clamp device. The clamp device may comprise a first arm comprising: an extended section comprising a mount at the distal end thereof; and a first jaw portion located at the distal end of the first arm. The clamp device may further comprise a second arm comprising: a fastening platform located at the proximal end of the second arm; and a second jaw portion located at the distal end of the second arm. The clamp device may further comprise engagement means comprising a bolt to engage the first arm with the second arm about a pivot point formed by the engagement means to provide relative rotation between: an open configuration whereby first jaw portion and second jaw portion are urged apart and a clamped configuration; and a clamped configuration whereby first jaw portion and second jaw portion are urged into clamping engagement with the patient's femur in use during a knee arthroplasty surgery. The clamp device may further comprise a fastening component comprising a fastening bolt and a fastening nut, adapted to engage the first and second arm into a locked clamped configuration about a bone of a patient during the surgical procedure. The method may comprise the further step of engaging the first and second jaw portion with a patient's bone to be stabilised.

The method may comprise the further step of applying a squeezing pressure to the clamp device to draw the top of the first and second arms together. The method may comprise the further step of locking the clamp device about the patient's bone. The method may comprise the further step of conducting a ligament balancing procedure with one hand of the surgeon holding a handle of the clamp device and the other hand of the surgeon manipulating the patient's leg thereby to impart a force to the clamp device indicative of the patient's ligament characteristics or morphology about the bone. The method may comprise the further step of determining ligament characteristics or morphology about the patient's joint or bone using the clamp device.

The surgical clamp device may comprise a sensor unit adapted to releasably engage the mount and configured to sense reactive forces applied to the joint or bone through the surgical clamp adapted for measuring quantitative force exchange data during an orthopaedic surgical procedure and to output the force exchange data; and the method may comprise the further step of receiving quantitative reactive force data from the sensor indicative of the patient's ligament characteristics or morphology about the patient's joint or bone.

According to a particular arrangement of the third aspect there is provided a method for determining knee ligament characteristics for a ligament balancing data during a surgical procedure comprising the steps of:
providing a surgical clamp device, the clamp device comprising:
   a first arm comprising:
      an extended section comprising a mount at the distal end thereof; and
      a first jaw portion located at the distal end of the first arm;
a second arm comprising:
   a fastening platform located at the proximal end of the second arm; and
      a second jaw portion located at the distal end of the second arm;
engagement means comprising a bolt to engage the first arm with the second arm about a pivot point formed by the engagement means to provide relative rotation between:
   an open configuration whereby first jaw portion and second jaw portion are urged apart and a clamped configuration; and
   a clamped configuration whereby first jaw portion and second jaw portion are urged into clamping engagement with the patient's femur in use during a knee arthroplasty surgery; and
a fastening component such as a fastening bolt and a fastening nut, adapted to engage the first and second arm into a locked clamped configuration about a bone of a patient during the surgical procedure;
engaging the first and second jaw portion with a patient's bone to be stabilised;
applying a squeezing pressure to the clamp device to draw the top of the first and second arms together;
locking the clamp device about the patient's bone;
conducting a ligament balancing procedure with one hand of the surgeon holding a handle of the clamp device and the other hand of the surgeon manipulating the patient's leg thereby to impart a force to the clamp device indicative of the patient's ligament characteristics or morphology about the bone; and
determining ligament characteristics or morphology about the patient's joint or bone using the clamp device.

The surgical clamp device may comprises a sensor unit adapted to releasably engage the mount and configured to sense reactive forces applied to the joint or bone through the surgical clamp adapted for measuring quantitative force exchange data during an orthopaedic surgical procedure and to output the force exchange data; wherein, the method further comprises receiving quantitative reactive force data from the sensor indicative of the patient's ligament characteristics or morphology about the patient's joint or bone.

The jaw portions of the clamp device may be located such that a curved section of the clamp device extends towards the lateral (outside) side of the patient's joint to avoid causing an obstruction to other tools forming part of the surgical procedure. The jaw portions of the clamp device may be locked in position about the patient's bone at a location of between about 5 to 10 cm from the distal end of the bone.

The data on the patient's ligament characteristics or morphology recorded from the clamp device may comprise force measurements in one or more planes. The force measurements may be characteristic of one or more angles of flexion in the one or more planes.

The data recorded from the clamp device may be used to appropriately tension the patient's ligaments during insertion of a prosthetic device to provide a balanced joint post-surgery.

The surgical procedure may be knee arthroplasty surgery and the patient's bone may be the femur. The surgical procedure may be elbow arthroplasty surgery and the patient's bone may be the humerus.

### BRIEF DESCRIPTION OF THE DRAWINGS

Notwithstanding any other forms which may fall within the scope of the present invention, a preferred embodiment/preferred embodiments of the invention will now be described, by way of example only, with reference to the accompanying drawings in which:

FIG. 1 shows a surgical clamp according to an embodiment of the present invention;

FIGS. 2 and 3 respectively show detail front and side plan views of a first arm of the surgical clamp of FIG. 1; and

FIGS. 4 and 5 respectively show detail front and side plan views of a second arm of the surgical clamp of FIG. 1;

FIG. 6 shows an exploded perspective view of the surgical clamp of FIG. 1;

FIGS. 7 and 8 respectively show front and side views of a sensor unit adapted for use with the surgical clamp of FIG. 1; and

FIG. 9 shows an exploded view of a sensor unit adapted for use with the clamp of FIG. 1.

In the drawings, like structures are referred to by like numerals throughout the several views. The drawings shown are not necessarily to scale, with emphasis instead generally being placed upon illustrating the principles of the present invention.

### Definitions

The following definitions are provided as general definitions and should in no way limit the scope of the present invention to those terms alone, but are put forth for a better understanding of the following description.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, additional terms are defined below. Furthermore, all definitions, as defined and used herein, should be understood to control over dictionary definitions, definitions in documents incorporated by reference, and/or ordinary meanings of the defined terms unless there is doubt as to the meaning of a particular term, in which case the common dictionary definition and/or common usage of the term will prevail.

For the purposes of the present invention, the following terms are defined below.

The articles “a” and “an” are used herein to refer to one or to more than one (i.e. to at least one) of the grammatical object of the article. By way of example, “an element” refers to one element or more than one element.

The term “about” is used herein to refer to quantities that vary by as much as 30%, preferably by as much as 20%, and more preferably by as much as 10% to a reference quantity. The use of the word ‘about’ to qualify a number is merely an express indication that the number is not to be construed as a precise value.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms: “including” or “which includes” or “that includes” as used herein is also an open term that also means including at least the elements/features that follow the term, but not excluding others. Thus, “including” is synonymous with and means “comprising”.

In the claims, as well as in the summary above and the description below, all transitional phrases such as “comprising.” “including.” “carrying.” “having.” “containing.” “involving.” “holding,” “composed of,” and the like are to be understood to be open-ended, i.e., to mean “including but not limited to”. Only the transitional phrases “consisting of” and “consisting essentially of” alone shall be closed or semi-closed transitional phrases, respectively.

The term, “real-time”, for example “displaying real-time data,” refers to the display of the data without intentional delay, given the processing limitations of the system and the time required to accurately measure the data. Similarly, a process occurring “in real time” refers to operation of the process without intentional delay or in which some kind of operation occurs simultaneously (or nearly simultaneously) with when it is happening.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A): in yet another embodiment, to at least one, optionally including more than one, A, and at least one, optionally including more than one, B (and optionally including other elements); etc.

For the purpose of this specification, where method steps are described in sequence, the sequence described herein does not necessarily mean that the steps are to be carried out in chronological order in that sequence, unless there is no other logical manner of interpreting the sequence.

In addition, where features or aspects of the invention are described in terms of Markush groups, those skilled in the art will recognise that the invention is also thereby described in terms of any individual member or subgroup of members of the Markush group.

### DETAILED DESCRIPTION

Modifications and variations such as would be apparent to the skilled addressee are considered to fall within the scope of the present invention. The present invention is not to be limited in scope by any of the specific embodiments described herein. These embodiments are intended for the purpose of exemplification only. Functionally equivalent products, formulations and methods are clearly within the scope of the invention as described herein. It will be understood by those skilled in the art that various changes in form and details may be made therein without departing from the spirit and scope of the invention as defined by the appended claims.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Such variations are not to be regarded as a departure from the disclosure, and all such modifications are intended to be included within the scope of the disclosure.

The method steps, processes, and operations described herein are not to be construed as necessarily requiring their performance in the particular order discussed or illustrated, unless specifically identified as an order of performance. It is also to be understood that additional or alternative steps may be employed.

Reference to positional descriptions and spatially relative terms, such as “inner,” “outer,” “beneath”, “below”, “lower”, “above”, “upper” and the like, are to be taken in context of the embodiments depicted in the figures, and are not to be taken as limiting the invention to the literal interpretation of the term but rather as would be understood by the skilled addressee.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

It will be understood that when an element is referred to as being “on”, “engaged”, “connected” or “coupled” to another element/layer, it may be directly on, engaged, connected or coupled to the other element/layer or intervening elements/layers may be present. Other words used to describe the relationship between elements/layers should be interpreted in a like fashion (e.g., “between”, “adjacent”). As used herein the term “and/or” includes any and all combinations of one or more of the associated listed items.

The terminology used herein is for the purpose of describing particular example embodiments only and is not intended to be limiting. The use of the singular Includes the plural unless specifically stated otherwise. As used herein, the singular forms “a”, “an” and “the” may be intended to include the plural forms as well, unless the context clearly indicates otherwise. The terms “comprise”, “comprises,” “comprising,” “including,” and “having,” or variations thereof are inclusive and therefore specify the presence of stated features, integers, steps, operations, elements, and/or components, but do not preclude the presence or addition of one or more other features, integers, steps, operations, elements, components, and/or groups thereof.

Correct ligament balancing results in a balanced joint, which in the context of a knee arthroplasty procedure, results in a balanced knee. A balanced knee comprises the following characteristics: a full range of movement; symmetrical medial-lateral balance at full extension and 90 degrees of flexion resulting in a rectangular tibiofemoral gap; correct valgus/varus alignment in both flexion and extension; balanced flexion-extension gap without medial-lateral tightness or laxity; a well-tracking patella during full motion; maximal flexion occurring with the patella reduced and without excessive rollback of the femur on the tibia; and correct rotational balance between the tibial and femoral components. Also, prosthetic wear and ligament balancing are intrinsically linked such that poor ligament balancing during the surgery leads inexorably to premature wear of the prosthetic implants (e.g. asymmetrical wear of the polyethylene insert component of the prosthesis) and ultimately failure of the prosthesis including osteolysis and prosthesis loosening requiring revision surgery.

The present invention provides an orthopaedic device 100 which can provide firm grasp of a bone and, with the addition of optional force sensor(s), simultaneously measure, record and transfer data on the forces and torques applied by a surgeon as part of an orthopaedic procedure, such as, for example, during a ligament balancing procedure as part of a total knee arthroplasty surgical procedure. In alternative embodiments, the orthopaedic device 100 may be also configured for different orthopaedic procedures which require the patient's bone structure to be held in a rigid or stable position to permit the surgeon to assess the patient's surrounding tissue structure with respect to a prosthetic implant such as, for example ligament balancing as discussed above. For example, minor modifications to the orthopaedic device 100 disclosed herein made be made as would be readily appreciated by the skilled addressee for additional surgical procedures including, for example, elbow arthroplasty procedures.

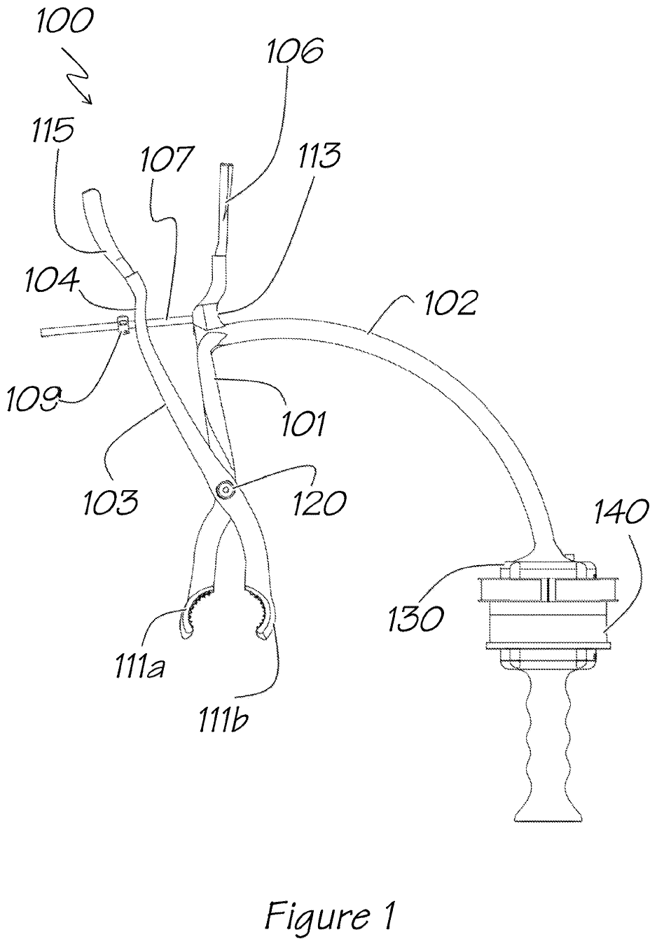

FIG. 1 shows a surgical clamp device 100 according to the present invention for use as a tool to securely grasp a bone of a patient during a surgical procedure and, with the addition of an optional attachable sensor unit 140, simultaneously record forces and torques applied by the surgeon as part of an orthopaedic procedure, such as a ligament balancing in total knee arthroplasty.

Clamp device 100 can be used both:
as a passive clamp to assist the surgeon and the surgical staff with current practices for ligament balancing by providing an improved means for maintain stability of the patient's leg during the surgeon's current ligament balancing procedures; and
a smart data measurement device, when used in conjunction with an optional force sensor unit 140 attachable to the clamp 100, the clamp can provide both the improved stability of the passive clamp; as well as display and record detailed reactive force data to the surgeon in real time during the surgical procedure, for example as part of a ligament balancing procedure, for explicit quantitative measurements of the ligament morphology before and after implantation of the surgical prosthesis.

The device 100 contains two main sections, a mechanical section capable to be used separately as a passive clamp device, and a sensor section adapted to be connected to the passive mechanical components for real-time quantitative reactive force data measurement as discussed in greater detail below.

### Mechanical Components

The mechanical section of device 100 is split into four separate functional pieces, and also contains two fastening pieces to allow rotation around a pivot point. All components of the mechanical system are detachable for compliance with sterilisation standards and further interchangeability of some functional pieces. The entire device can also be mirrored to cater for either the left or the right leg of the patient. In preferred embodiments, the clamp 100 is designed to be manufactured in separate, connectable sections, suitable for rapid manufacturing methods such as 3D printing, either in synthetic materials or 3D metal printing processes. For example, the respective handles 16 and 115 of the first and second arms 101 and 103 as shown in FIG. 1 may be manufactured independently of the arms and connected thereafter to reduce the size of the component to be manufactured by each step in the process.

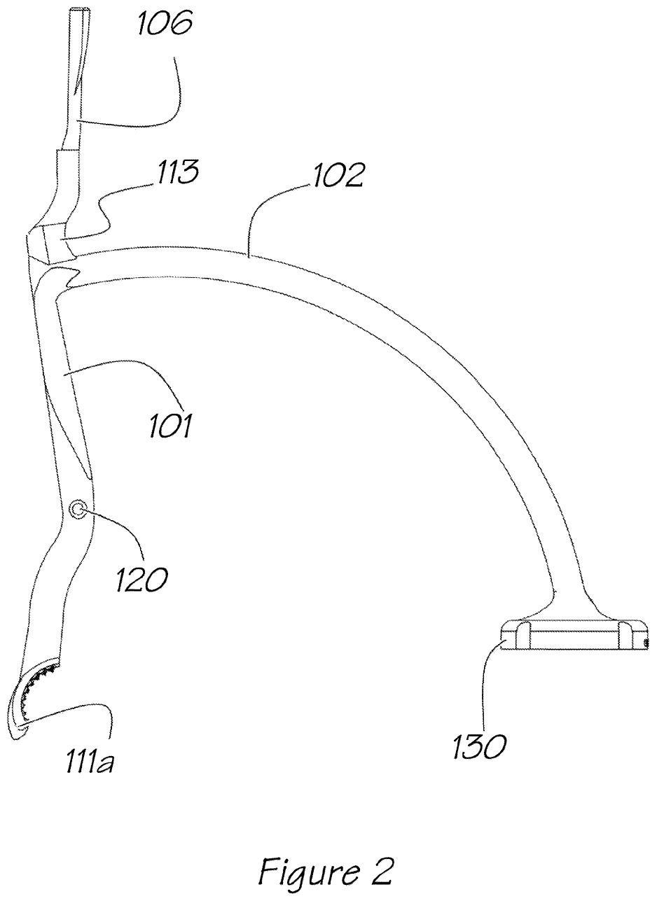

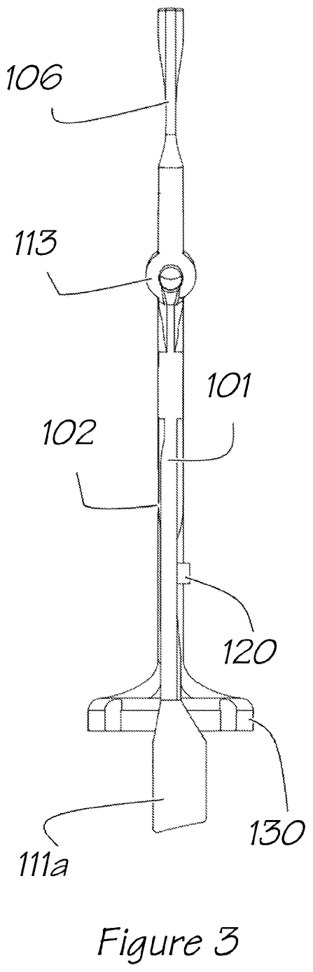

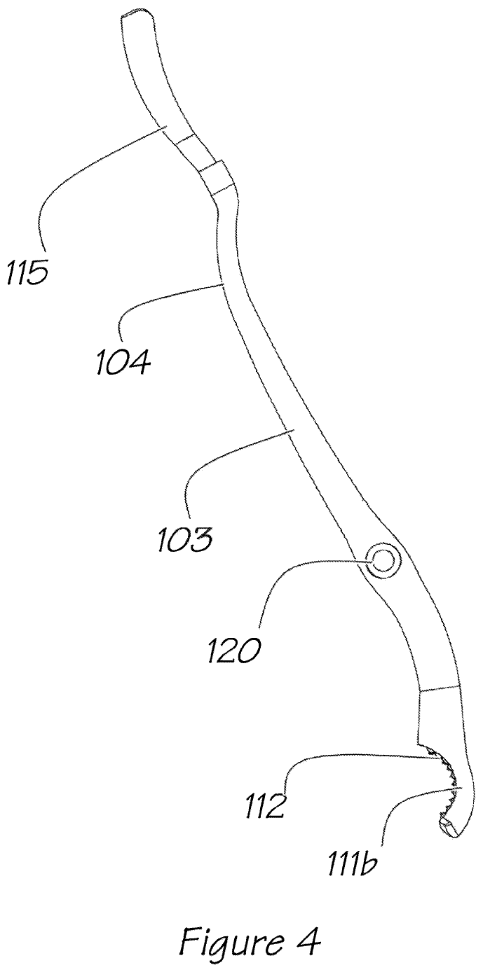

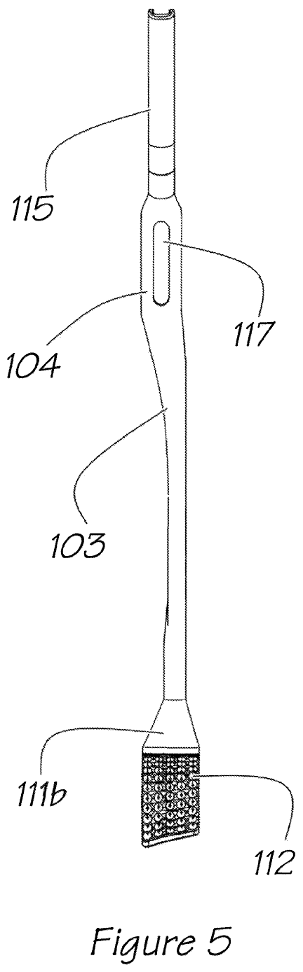

The functional components of clamp 100 include a first arm 101 and a second arm 103, a spherical headed, bolt 107 to connect arms 101 and 103 to each other, and a bespoke nut 109 to lock the arms 101 and 103 in place at a desired position during use. FIG. 2 and FIG. 3 respectively show detail front and side plan views of first arm 101 of clamp 100 as depicted in FIG. 1. FIG. 4 and FIG. 5 respectively show detail front and side plan views of second arm 103 as depicted in FIG. 1. Jaws 111 of clamp 100 comprising first jaw portion 111a and second jaw portion 111b respectively at the distal ends of arms 101 and 103 are designed specifically to match the morphology of the patient's bone at a location where attachment of the clamp will not interfere with the surgical procedure, such as the distal femur shaft in the case of knee arthroplasty. The clamp 100 features a mechanical class 1 lever system, which allows the clamp 100 to adjust for a firm grip in the desired area of the femur shaft, so it can be suited to a range of patients.

First arm 101 of clamp 100 comprises an extended curved section 102 adapted to be positioned, when in use, such that is extends towards the lateral (outside) side of the patient's knee to avoid causing an obstruction to other tools forming part of the surgical procedure. Curved section 102 is curved for ergonomic compactness, allowance for the soft tissue envelope of the thigh, and the minimisation of strain throughout clamp 100. The end of curved section 102 comprises a mount 130 adapted to receive either:
a passive handle device (not shown) for the surgeon to manipulate the patient's limb during the procedure; or
an active handle comprising a sensor unit 140 for measurement of the forces and torques applied to the patient's femur during the ligament balancing procedure as discussed below. In preferred embodiments of the clamp device 100, the sensor unit 140 comprises one or more multiaxial sensor(s), adapted to record reactive forces from the patient's ligaments whilst the surgeon manipulates the limb.

The passive or active handle may be interchangeable with, for example, varying handle designs to provide options to the surgeon and/or surgical assistant to choose a handle design have a preferred grip structure (e.g. contour moulding 149 across the surface of handle).

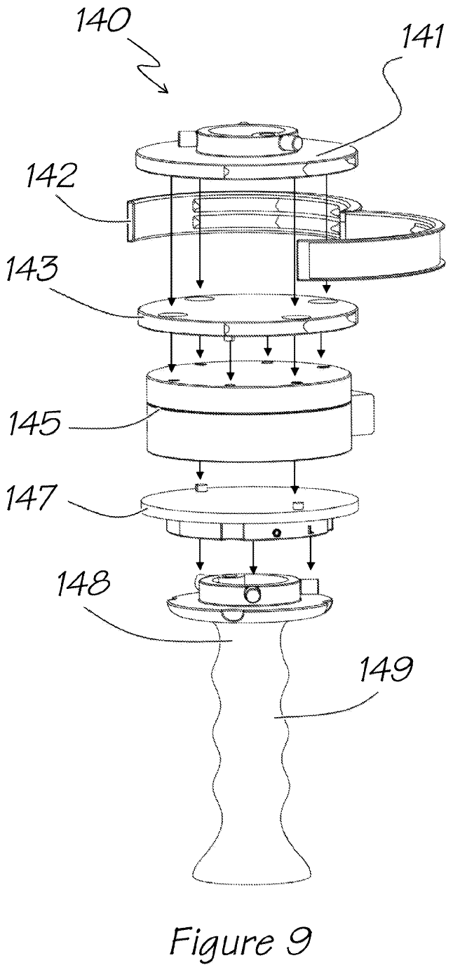

In other embodiments, the handle is permanently fixed to the extended curved arm 102. In such embodiments, the handle is configured to receive a releasably attachable sensor unit (not shown) similar to sensor unit 140 of FIG. 9 to allow the passive handle to be converted to an active handle comprising a plurality of force sensors to measure and provide real-time or near-real-time force data to the surgeon or surgical assistant.

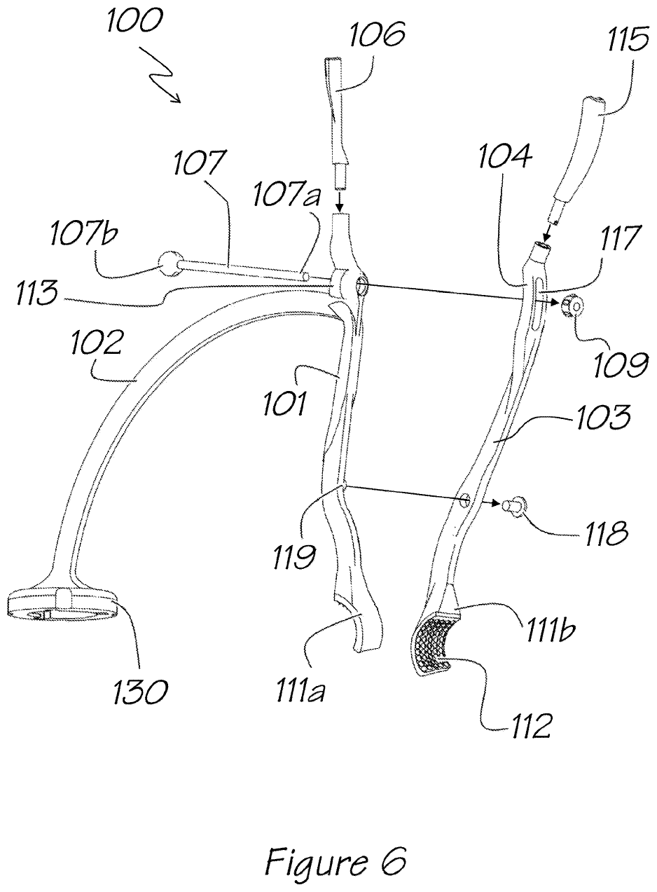

The first arm 101 comprises a curved handle 106 used to assist with holding the clamp 100 in place before it is locked in position by adjustment of a fastening nut 109 engaged with bolt 107 which is (releasably) engaged with first arm 101 and passing through a slot 117 (as seen in FIG. 6) of second arm 103.

Second arm 103 of clamp 100 is provided in a slim design to minimise obstructions to the surgical procedure. Second arm 103 comprise a long, slightly curved fastening platform 104 which includes a slot 117 (as seen in FIG. 5) removed in the centre of fastening platform 104 allowing for the clamp 100 to be operated and locked at a range of angles, which corresponds to a large range of femur bone sizes encountered by orthopaedic surgeons. Similarly, second arm 103 comprises a curved handle section 115 for use by the surgeon to hold clamp 100 in place before it is locked in position by adjustment of fastening nut 109.

Bolt 107 is provided to engage both arms 101 and 103 to enable clamp 100 to be locked into a closed/clamped position about the patient's femur during use. Rod 107 is detachable from clamp 100 to facilitate easier cleaning in a suitable cleaning device, for example a hospital-grade autoclave. Bolt 107 comprises a spherical head 107b adapted to engage with a hemispherical receptacle 113 in the distal end of arm 101 to permit a slight rotation on all axes. This rotation enables a secure fit on a range of different bone sizes while simultaneously increasing resistance to torsional forces subjected to the clamp. Bolt 107 slides through a larger slot 117 on the fastening arm (i.e. second arm 103), to link the two arms 101 and 103 together. Bolt 107 is used to lock the jaws 111a and 111b of clamp 100 into a clamped position on the patient's femur by screwing in matching nut 109 along bolt 107 against fastening platform 104 by hand.

Arms 101 and 103 are connected by a nut 119 and bolt 118 so as to form a pivot point 120 (shown in FIG. 1) to permit relative rotation of first and second arms 101 and 103. In a particular embodiment small detachable bolt 118 is screwed into a cylindrical barrel nut 119. Alternative functionally equivalent attachment means to form pivot point 120 would also be readily appreciated by the skilled addressee.

In the example context of a TKR surgical procedure, to operate clamp 100, initially, fastening nut 109 is screwed to the distal end 107a of bolt 107 to allow for maximum pivoting movement of the clamp arms about pivot point 120 and opening of jaws 111 before the clamp is inserted and engaged with the patient's femur bone. This allows the surgeon to open jaws 111 of clamp 100 to its maximum extent to provide ample space between the jaws 111 to fit clamp 100 around the patient's femur without unnecessary tissue damage. Depending on the size of the patient's femur bone, the jaws are placed a suitable distance along the distal end of the femur (for example about 5 to 10 cm from the distal head of the femur) so the jaws 111 match the femur morphology as closely as practicable. The clamp 100 is then operated using a squeezing pressure applied by the surgeon against curved handles 106 and 115 (and thus, fastening platform 104), to draw the top of clamp arms 101 and 103 together. Once a snug fit to the patient's femur has been achieved, the surgeon fastens clamp 100 by screwing the fastening nut 109 in to meet arm 103 against fastening platform 104, such that clamp 100 is secured tightly around the patient's femur.

The jaws 111 of clamp 100 are rounded in shape to fit with the natural shape of the patient's bone to which the clamp 100 is designed to engage. In use in TKR surgery, when locked into position with the patient's femur, clamp 100 preferably assumes a position where the locking arm 101 leans between about 15 to 25 degrees, optimally about 20-degrees, from the vertical towards the medial side, such that pivot point 120 is substantially above the centre of the femur. This placement of clamp 100 ensures alignment with surgical cuts to the femur performed during the knee arthroplasty procedure and to allow access and visibility for the surgeon while ensuring no additional/unnecessary tissue damage is inflicted upon the patient. The interior concave surface of the clamp jaws 111 features an extruded pattern 112 to improve grip of the jaws and reduce slippage about the patient's femur during use.

In use, once secured to the patient's femur, the surgeon or assistant holds clamp 100 in one hand by a handle (not shown) or sensor unit 140 securely attached to the mount 130 located at the distal end of extended curved section 102 and manipulates the patient's lower leg with their other hand whilst assessing the reactive/resistive forces of the patient's limb structures, particularly the patient's ligament structures about the joint (e.g. knee, elbow, etc) which is the subject of the surgical procedure.

All components of the mechanical system are detachable for compliance with sterilisation standards. The entire device can be mirrored to cater for either the left or the right leg of the patient.

### Sensor Components

Clamp 100 further comprises an optional sensor unit 140 designed to engage with mount 130 located at the distal end of curved section 102 of first arm 101, and measure the forces applied by the surgeon or a surgical assistant on the joint during the ligament balancing process. The sensor unit 140 is adapted to releasably engage with sensor housing unit 130 of clamp 100 to permit clamp device 100 to be used interchangeably as a passive clamping device or as a smart sensing device for real or near real time measurement and reporting of force data to the surgeon. Sensor unit 140 contains 2 main areas, the sensor body comprising of a multiaxial force torque sensor 145 fitted to a pair of custom ribbed sterilisable mounting plates 141 and 143, and handle 148. To conform with sterilisation standards, a sterile plastic sleeve (not shown) is fitted between plates 141 and 143 before fixation to the mount 130 on the distal end of the mechanical extension 102. The handle 148 can then be held by the surgeon, and the clamp 100 is then ready for operation. The normal steps for ligament balancing in, for example, a TKR procedure, are then undertaken by the surgeon during the surgical procedure, but with one hand holding the device handle, and the other applying forces on the lower leg. This allows the patient's upper leg to remain stable throughout the balancing procedure and improve control of the contextual environment in which quantitative data is measured by sensor unit 140, rather than just relying upon the surgeon's estimation.

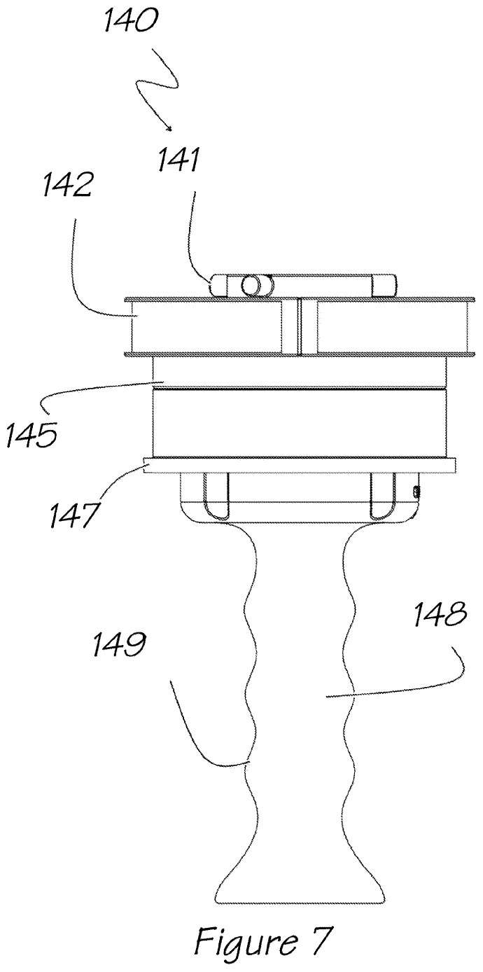

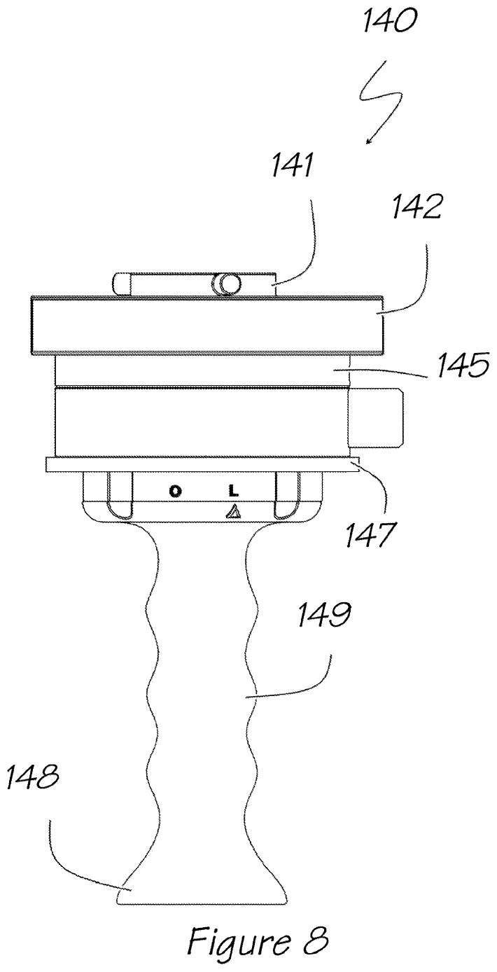

FIGS. 7 and 8 respectively show front and side plan views, whereas FIG. 9 shows a perspective view, of a particular embodiment of an example sensor unit 140 adapted for use with clamp 100. Referring particularly to FIG. 9, the first ribbed plate 141 is locked into the extension of the extended curved section 102 of first arm 101, for example by a twist-lock mechanism. A sterile collar 142 releasably engages ribbed plate 141 and a second ribbed plate 143 to tightly secure a sterile plastic sleeve (not shown) between plates 141 and 143. The fixation of the sterile sleeve provides secure coverage over the distal end of sensor unit 140. Plate 143 is screwed into the top side of a multi-axial force torque sensor 145. The sensor 145 is screwed into sensor mounting plate 147 of which is releasably engaged with handle 148.

Sensor 145, in use, provides force and torque data to the sensor unit 140 to facilitate data read-out of the force and torque applied by a surgeon during the ligament balancing procedure as part of a surgical procedure, e.g. knee replacement, on a patient.

Handle 148 preferably comprises contour moulding 149 across the surface of handle 148 for an ergonomic fit in the surgeon's hand. This allows for greater control accuracy and physical stabilisation of the device 100 during ligament balancing. When handle 148 is engaged with sensor mounting plate 147 within sensor unit 140, reactive forces are placed on the handle 148 for detection by the multiaxial force and torque sensor 145. These reactive forces can then be calibrated accordingly to give a real-time quantitative and qualitative output indication of the force and torque applied to the patient's knee. This provides a quantitative measurement of the ligament properties before and after insertion of the knee prostheses to ensure adequate ligament balancing has been achieved to, as closely as possible, mimic the patient's ligament structure and function prior to the surgery. Handle 148 is further adapted to releasably engage mount 130 of arm 101 in an alternative configuration where sensor unit 140 is optional such that clamp 140 may serve as a purely mechanical surgical stabilisation device for ligament balancing and when quantitative data of the ligament balancing procedure is not required.

The sensor 145 optionally further comprises a data read out port that facilitates the transmission of raw data via cabled or wireless methods to an external module (not shown) for processing and presentation of the sensed data to the surgeon. For example, in particular arrangements, handle 148 of sensor device 140 may be hollow to provide electrical wiring connections between the multiaxial force and torque sensor 145 and a data read-out port (not shown). In alternate arrangements, sensor unit 140 may optionally include a wireless connection module adapted to receive data from each of the plurality of sensors 145 and wirelessly transmit the data in real-time to a wireless receiving module which receives the transmitted data and presents it to the surgeon in real-time, or near real-time. A processor may also optionally be provided, either comprised within handle 149 or externally (for example, connected to a wireless receiving module) to conduct analysis calculations and procedures on the sensed data prior to presentation of the raw sensor data and/or additional calculated data parameters to the surgeon in real-time or near-real-time.

All components of the clamp 100 described above, including, at least, the first and second arms 101 and 103 arm, sensor unit 140, fastening bolt 107 and fastening nut 109, as well as pivot point fasteners 118 and 119—are preferably all made of stainless steel and can be fabricated from other substitute sterilisable materials such as titanium which have similar hardness and elasticity characteristics. The components of clamp 100 may be fabricated via 3D printing, CNC machining, or die casting methods as would be appreciated by the skilled addressee.

### Embodiments

Reference throughout this specification to “one embodiment”, “an embodiment”, “one arrangement” or “an arrangement” means that a particular feature, structure or characteristic described in connection with the embodiment/arrangement is included in at least one embodiment/arrangement of the present invention. Thus, appearances of the phrases “in one embodiment/arrangement” or “in an embodiment/arrangement” in various places throughout this specification are not necessarily all referring to the same embodiment/arrangement but may. Furthermore, the particular features, structures or characteristics may be combined in any suitable manner, as would be apparent to one of ordinary skill in the art from this disclosure, in one or more embodiments/arrangements.

Similarly, it should be appreciated that in the above description of example embodiments/arrangements of the invention, various features of the invention are sometimes grouped together in a single embodiment/arrangement, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment/arrangement. Thus, the claims following the Detailed Description are hereby expressly incorporated into this Detailed Description, with each claim standing on its own as a separate embodiment/arrangement of this invention.

Furthermore, while some embodiments/arrangements described herein include some but not other features included in other embodiments/arrangements, combinations of features of different embodiments/arrangements are meant to be within the scope of the invention, and form different embodiments/arrangements, as would be understood by those in the art. For example, in the following claims, any of the claimed embodiments/arrangements can be used in any combination.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Different Instances of Objects

As used herein, unless otherwise specified the use of the ordinal adjectives “first”, “second”, “third”, etc., to describe a common object, merely indicate that different instances of like objects are being referred to, and are not intended to imply that the objects so described must be in a given sequence, either temporally, spatially, in ranking, or in any other manner.

### Comprising and Including:

In the claims which follow and in the preceding description of the invention, except where the context requires otherwise due to express language or necessary implication, the word “comprise” or variations such as “comprises” or “comprising” are used in an inclusive sense, i.e., to specify the presence of the stated features but not to preclude the presence or addition of further features in various embodiments of the Invention.

Any one of the terms: “including” or “which includes” or “that includes” as used herein is also an open term that also means “including at least” the elements/features that follow the term, but not excluding others. Thus, including is synonymous with and means comprising.

### Scope of Invention

Thus, while there has been described what are believed to be the preferred arrangements of the invention, those skilled in the art will recognize that other and further modifications may be made thereto without departing from the spirit of the invention, and it is intended to claim all such changes and modifications as fall within the scope of the invention. Functionality may be added or deleted from the block diagrams and operations may be interchanged among functional blocks. Steps may be added or deleted to methods described within the scope of the present invention.

Although the invention has been described with reference to specific examples, it will be appreciated by those skilled in the art that the invention may be embodied in many other forms.

### INDUSTRIAL APPLICABILITY

It is apparent from the above, that the arrangements described are applicable to the mobile device industries, specifically for methods and systems for distributing digital media via mobile devices.

It will be appreciated that the methods/apparatus/devices/systems described/illustrated above at least substantially provide a surgical clamp for use in ligament balancing procedures during knee arthroplasty surgery.

The surgical clamp and ligament balancing methods described herein, and/or shown in the drawings, are presented by way of example only and are not limiting as to the scope of the invention. Unless otherwise specifically stated, individual aspects and components of the surgical clamp, or steps of the ligament balancing method, may be modified, or may have been substituted therefore known equivalents, or as yet unknown substitutes such as may be developed in the future or such as may be found to be acceptable substitutes in the future. The surgical clamp and ligament balancing method may also be modified for a variety of applications while remaining within the scope and spirit of the claimed invention, since the range of potential applications is great, and since it is intended that the present surgical clamp and methods be adaptable to many such variations.

The claims defining the invention are as follows:

1. A surgical clamp for providing a stable mechanical grasp and stabilisation of bone during an orthopaedic surgical procedure, comprising:
a first arm comprising:
an extended section comprising a mount at the distal end thereof; and
a first jaw portion located at the distal end of the first arm;
a second arm comprising:
a fastening platform located at the proximal end of the second arm; and
a second jaw portion located at the distal end of the second arm;
engagement means comprising a bolt to engage the first arm with the second arm about a pivot point formed by the engagement means to provide relative rotation of the first and second arms between:
an open configuration whereby first jaw portion and second jaw portion are urged apart; and
a clamped configuration whereby first jaw portion and second jaw portion are urged into clamping engagement with the patient's bone in use during the surgical procedure;
a fastening component comprising a fastening bolt and a fastening nut, adapted to engage the first and second arm into a locked clamped configuration about the bone of a patient during the surgical procedure; and
a handle adapted to be gripped by a surgical assistance for stabilisation of the patient's joint or bone during the surgical procedure.

2. A surgical clamp as claimed in claim Error! Reference source not found. w herein the handle comprises:
a sensor unit adapted to releasably engage the mount and configured to sense reactive forces applied to the bone through the surgical clamp adapted for measuring quantitative force exchange data during an orthopaedic surgical procedure and to output the force exchange data.

3. A surgical clamp as claimed in claim 2 wherein the sensor unit comprises;
a multiaxial sensor adapted to provide quantitative reactive force and/or torque measurements, as may be generated during an intermediate procedure of the surgical procedure; and
a fixture component to ensure the sterile state of non-mechanical components including the said multiaxial sensor.

4. A surgical clamp as claimed in claim 3 wherein the intermediate procedure is a ligament rebalancing procedure.

5. A surgical clamp as claimed in claim 3 wherein the fixture component comprises:
a pair of ribbed plates; and
a securing component adapted to releasably engage the pair of ribbed plates.

6. A surgical clamp as claimed in claim 5 wherein the securing component comprises a sterile collar.

7. A surgical clamp as claimed in any one of the preceding claims wherein the handle is interchangeable.

8. A surgical clamp as claimed in any one of the preceding claims wherein an inner face of the first and second jaw portions are adapted to approximately match the shape of the patient's bone.

9. A surgical clamp as claimed in any one of the preceding claims wherein an inner face of each of the first and second jaw portions is conditioned with a surface to facilitate secure clamping engagement with the patient's bone.

10. A surgical clamp as claimed in claim 9 comprising contouring of the said inner face.

11. A surgical clamp as claimed in any one of the preceding claims wherein:
the first arm comprises a receptacle located at the proximal end thereof;
the second arm comprises a fastening platform at the proximal end thereof comprising an elongate slot;
the proximal end of the fastening bolt comprises a stop adapted to engage the receptacle;
the distal end of the fastening bolt is adapted to pass though the elongate slot; and
wherein the fastening nut is adapted to engage the fastening bolt with the fastening platform thereby to secure the surgical clamp into a clamped locked configuration.

12. A surgical clamp as claimed in any one of the preceding claims wherein the sensor unit provides output data quantitatively representative of the forces and torques applied to the stabilised bone during surgical procedure.

13. A surgical clamp as claimed in any one of the preceding claims wherein the surgical procedure is knee arthroplasty surgery and the patient's bone is the femur.

14. A surgical clamp as claimed in any one of claims 1 to 12 wherein the surgical procedure is elbow arthroplasty surgery and the patient's bone is the humerus.

15. An active surgical clamp for providing a stable mechanical grasp of bone and simultaneously measuring quantitative force exchange data during an orthopaedic surgical procedure, comprising:
the surgical clamp of claim 1; and
a sensor unit adapted to releasably engage the mount and configured to sense reactive forces applied to the bone through the surgical clamp adapted for measuring quantitative force exchange data during an orthopaedic surgical procedure and to output the force exchange data.

16. A method for determining knee ligament characteristics for a ligament balancing data during a surgical procedure comprising the steps of:
providing a surgical clamp device, the clamp device comprising:
a first arm comprising:
an extended section comprising a mount at the distal end thereof; and
a first jaw portion located at the distal end of the first arm;
a second arm comprising:
a fastening platform located at the proximal end of the second arm; and
a second jaw portion located at the distal end of the second arm;
engagement means comprising a bolt to engage the first arm with the second arm about a pivot point formed by the engagement means to provide relative rotation between:
an open configuration whereby first jaw portion and second jaw portion are urged apart and a clamped configuration; and
a clamped configuration whereby first jaw portion and second jaw portion are urged into clamping engagement with the patient's femur in use during a knee arthroplasty surgery; and
a fastening component comprising a fastening bolt and a fastening nut, adapted to engage the first and second arm into a locked clamped configuration about a bone of a patient during the surgical procedure;
engaging the first and second jaw portion with a patient's bone to be stabilised;
applying a squeezing pressure to the clamp device to draw the top of the first and second arms together;
locking the clamp device about the patient's bone;
conducting a ligament balancing procedure with one hand of the surgeon holding a handle of the clamp device and the other hand of the surgeon manipulating the patient's leg thereby to impart a force to the clamp device indicative of the patient's ligament characteristics or morphology about the bone; and
determining ligament characteristics or morphology about the patient's joint or bone using the clamp device.

17. A method as claimed in claim 16, wherein the surgical clamp device comprises a sensor unit adapted to releasably engage the mount and configured to sense reactive forces applied to the joint or bone through the surgical clamp adapted for measuring quantitative force exchange data during an orthopaedic surgical procedure and to output the force exchange data;
wherein, the method further comprises receiving quantitative reactive force data from the sensor indicative of the patient's ligament characteristics or morphology about the patient's joint or bone.

18. A method as claimed in either claim 16 or claim 17, wherein the jaw portions of the clamp device are located such that a curved section of the clamp device extends towards the lateral (outside) side of the patient's joint to avoid causing an obstruction to other tools forming part of the surgical procedure.

19. A method as claimed in any one of claims 16 to 18, wherein the jaw portions of the clamp device are locked in position about the patient's bone at a location of between about 5 to 10 cm from the distal end of the bone.

20. A method as claimed in any one of claims 16 to 19, wherein the data on the patient's ligament characteristics or morphology recorded from the clamp device comprises force measurements in one or more planes.

21. A method as claimed in claim 20, wherein the force measurements are characteristic of one or more angles of flexion in the one or more planes.

22. A method as claimed in any one of claims 16 to 21, wherein the data recorded from the clamp device is used to appropriately tension the patient's ligaments during insertion of a prosthetic device to provide a balanced joint post-surgery.

23. A method as claimed in any one of claims 16 to 22, wherein the surgical procedure is knee arthroplasty surgery and the patient's bone is the femur.

24. A method as claimed in any one of claims 16 to 22, wherein the surgical procedure is elbow arthroplasty surgery and the patient's bone is the humerus.
